(19) United States
(12) Patent Application Publication
(10) Pub. No.: US 2007/0196436 A1
(43) Pub. Date: Aug. 23, 2007

(54) PROCESS FOR PREPARING AN EDIBLE COMPOSITION COMPRISING STEROIDAL GLYCOSIDES

(75) Inventors: Salomon Leendert ABRAHAMSE, 3133 AT Vlaardingen (NL); Ivo Antonius van AMERONGEN, 3133 AT Vlaardingen (NL); Kevin John POVEY, Purfleet, Essex RM19 1SD (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco, Inc., d/b/a Unilever, Englewood Cliffs, NJ (US)

(21) Appl. No.: 11/279,624

(22) Filed: Apr. 13, 2006

(30) Foreign Application Priority Data
Feb. 23, 2006 (EP) EP06110333

(51) Int. Cl. A61K 31/704; A61K 47/00

(52) U.S. Cl. 424/439; 514/26

(57) ABSTRACT

A process for preparing an edible aqueous dispersion of one or more steroidal glycosides, the edible dispersion comprising one or more steroidal glycoside, and a food product comprising the dispersion, as well as methods of controlling body weight or obesity by ingesting the food product.

### FIELD OF THE INVENTION

The present invention relates generally to the field of edible compositions comprising steroidal glycosides. More in particular, it relates to a process for preparing an edible composition comprising steroidal glycosides with good dispersibility. Preferably, the compositions comprise steroidal glycosides of the Asclepiadaceae family, in particular of the Hoodia genus (formerly the Hoodia and Trichocaulon genera), which can be used for example in weight management products

### BACKGROUND OF THE INVENTION

Extracts obtainable from plants of the Asclepiadaceae family, particularly the Hoodia genus (formerly the Hoodia and Trichocaulon genera) have been shown to have an appetite suppressant or satiety activity and are potentially useful in weight management products. U.S. Pat. No. 6,376,657 discloses that these plants contain steroidal glycosides having the formula 1: 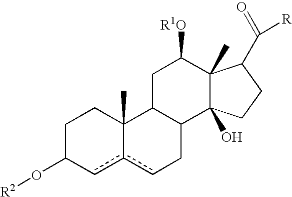
wherein
R=alkyl;
R¹=H, alkyl, tiglyol, benzoyl or any other organic ester group;
R²=H or one or more 6-deoxy carbohydrates, or one or more 2,6-dideoxy carbohydrates, or glucose molecules, or combinations thereof; and wherein the broken lines indicate the optional presence of a further bond between carbon atoms C4 and C5 or between carbon atoms C5 and C6.

U.S. Pat. No. 6,376,657 also discloses a process to extract the steroidal glycoside having the formula 1 from plants of the Asclepiadaceae family, involving treating plant material with a solvent to extract a fraction having appetite suppressant activity, separating the extraction solution from the rest of the plant material, removing the solvent from the extraction solution and recovering the extract. The solvents specifically mentioned to perform the extraction are one or more of methylene chloride (dichloromethane), water, methanol, hexane, ethyl acetate or mixtures thereof. An alternative method to obtain an extract is disclosed by separating plant sap from the plant solid material.

WO2005/116049 (Unilever) discloses that steroidal glycosides can be extracted or separated from undesirable components present in plant material of the Asclepiadaceae (Hoodia) family by means of liquid or supercritical carbon dioxide.

The present inventors have found that it is difficult to formulate suitable food products comprising such steroidal glycosides or extracts, especially of the compounds having formula 1 from plants of the Asclepiadaceae family, because the extracts and the pure compounds are solid at room temperature. Moreover, they are not soluble in water and poorly soluble in edible fats. As a consequence, these materials are very difficult to disperse in a food product. The poor solubility of steroidal glycosides in oil or water leads to the formation of large solid aggregates, which impart a gritty texture and give a poor appearance of the foods. Furthermore, the poor dispersability of the material makes cleaning of the food processing equipment difficult and can lead to problems when the material is mixed with or dispersed in other food ingredients. Finally, even when acceptable food products can be produced, the bioavailability of the active compounds is not optimal.

Accordingly, there is a need for a process for preparing an edible aqueous dispersion of one or more steroidal glycosides, especially physically stable dispersions of the extracts or the pure compounds having formula 1, from plants of the Asclepiadaceae family, which process overcomes one or more of these disadvantages. A further object of the invention is to provide an edible dispersion of steroidal glycosides and food products comprising the dispersion.

We have now surprisingly found that these and other objects of the invention may be achieved by the process for preparing an edible aqueous dispersion of one or more steroidal glycosides, comprising the steps of:
(a) preparing a composition comprising at least 1% by weight steroidal glycosides,
(b) dissolving an emulsifier having a HLB (hydrophilic lipophilic balance) value between 7 and 30 in a solvent, and
(c) thoroughly homogenising the composition comprising the steroidal glycosides for about 1 to 10 minutes in the solution of the solvent and the emulsifier at a temperature of 20 to 200° C. to obtain a dispersion, whereby said steroidal glycosides are dispersed to a mean particle size (D3,2) of less than 15 micrometer, wherein the ratio (w/w) of the steroidal glycosides to the emulsifier in said edible dispersion is less than 10:1.

These dispersions are stable and can be used in a simple and particularly efficient method for preparing food products comprising steroidal glycosides, such as appetite suppressant compositions or satiety compositions.

### SUMMARY OF THE INVENTION

In a first aspect, the present invention relates to a process for preparing an edible aqueous dispersion of one or more steroidal glycosides, comprising the steps of:
(a) preparing a composition comprising at least 1% by weight steroidal glycosides,
(b) dissolving an emulsifier having a HLB (hydrophilic lipophilic balance) value between 7 and 30 in a solvent, and
(c) thoroughly homogenising the composition comprising the steroidal glycosides for 1 to 10 minutes in the solution of the solvent and the emulsifier at a temperature of 20 to 200° C. to obtain a dispersion, whereby said steroidal glycosides are dispersed to a mean particle size (D3,2) of less than 15 micrometer, wherein the ratio (w/w) of the steroidal glycosides to the emulsifier in said edible dispersion is less than 10:1.

In a second aspect, the invention relates to an edible aqueous dispersion, and in a third aspect, the invention relates to a food product comprising the edible dispersion. In a fourth aspect, the invention also includes methods of managing body weight, suppressing appetite and controlling obesity, the method comprising ingesting (eating or drinking) the inventive food products.

### DETAILED DESCRIPTION OF THE INVENTION

Except in the operating and comparative examples, or where otherwise explicitly indicated, all numbers in this description indicating amounts of material or conditions of reaction, physical properties of materials and/or use are to be understood as modified by the word “about.”

It should be noted that in specifying any range of concentration, any particular upper concentration can be associated with any particular lower concentration.

For the avoidance of doubt the word “comprising” is intended to mean “including” but not necessarily “consisting of” or “composed of.” In other words, the listed steps or options need not be exhaustive.

The first aspect of the present invention is a process for preparing a stable edible aqueous dispersion of one or more steroidal glycosides. The steroidal glycosides are preferably obtained from plants, especially plants of the Asclepiadaceae family. In the process of the invention, a composition comprising at least 1% by weight of steroidal glycosides is dispersed in a solution of an emulsifier. Preferably, this first composition is an extract of the plants, more preferably an extract comprising at least 10% steroidal glycosides. U.S. Pat. No. 6,376,657, incorporated by reference herein, describes the preparation of a suitable extract comprising steroidal glycosides from the genus Hoodia, said steroidal glycoside having appetite suppressant activity. “Extract” as used herein includes liquid, solid or spray-dried forms of extracts, sap, which also may be purified, partially purified, concentrated and/or fractionated. Besides the extract, the first composition may be in the form of otherwise concentrated preparation, e.g. a dried plant or otherwise concentrated plant preparation, as long as it contains at least 1% by weight of steroidal glycosides.

The first composition comprising at least 1% by weight of steroidal glycosides is dispersed in a solution of an emulsifier, at a temperature of 20 to 200° C. This solution is prepared by dissolving an emulsifier having a HLB (hydrophilic lipophilic balance) value between 7 and 30 in a suitable solvent at a temperature of 20 to 200° C. The solvent is preferably water, but if the melting point of the composition comprising at the steroidal glycosides or the plant extract is above 100° C., one can also use food-grade oils, such as triglycerides from animal or vegetable origin. Vegetable oils such as olive oil, sunflower oil or palm oil are preferred.

Subsequently, the composition comprising the steroidal glycosides is thoroughly homogenised for 1 to 10 minutes in the solution of the emulsifier at a temperature of 20 to 200° C., preferably from 50 to 120° C., more preferably from 80 to 110° C. The amount of steroidal glycosides in the solution is from 0.001 to 10% by weight. When the melting temperature is reached, the mixture is thoroughly homogenised for about 1 to 10 minutes by means of a suitable mixer or vortex stirrer. Suitable equipment includes the Silverson (at about 4,500 to 6,000 rpm), Ultra-turrax, (High pressure) piston homogenisors, Colloid mills or Micro-fluidisers. Subsequently, the mixture is cooled to a temperature below 40° C. (cooling is optional depending on the temperature of homogenization) and the obtained dispersion of steroidal glycosides has the desired mean particle size (D3,2) of less than 15 micrometer. The small particle size causes the dispersions to be especially stable and smooth in texture. The aqueous dispersions of the invention comprise a highly hydrophilic emulsifier having a HLB (hydrophilic lipophilic balance) value between 7 and 30. Examples of preferred emulsifiers that can be used in the present invention are SSL (sodium stearoyl lactylate), Tween 60 (POE (20) sorbitan monostearate), Bolec MT (enzymatically hydrolysed Lecithin) and L1695 (lauric ester of sucrose ex Mitsubishi-Kasei Food Corp.). The preferred emulsifier is SSL.

The weight ratio (w/w) of the steroidal glycosides to the emulsifier in the edible dispersion is less than 10:1. Preferably it is from 10:1 to 1:1,000, more preferably from 10:1 to 1:100.

In the edible aqueous dispersions of the present invention, the steroidal glycosides are preferably dispersed to a mean particle size of less than 15 micrometer, more preferably less than 10 micrometer, more preferably less than 8 micrometer. Most preferably, the mean particle size is from 0.3 to 8 micrometer. By mean particle size we mean in the context of the present invention the D3.2 particle size, i.e. the average diameter expressed as the volume/surface average. Particle sizes can be measured using well-known techniques and equipment, such as laser diffraction spectrometry with the Helium-Neon Laser Optical System from Sympatec GmbH (Clausthal-Zellerfield, Germany). The small particle size was found to be preferred for obtaining an acceptable mouthfeel and appearance of food products.

The process of the present invention is preferably applied to an extract comprising the steroidal glycosides from plants of the Asclepiadaceae family, more preferably from the genus Hoodia.

In a particularly advantageous embodiment of the invention, the steroidal glycoside is selected from the group consisting of appetite suppressing steroidal glycosides that can be extracted from plants of the genus Trichocaulon or of the genus Hoodia, derivatives, salts and analogues of said steroidal glycosides and mixtures thereof.

According to an especially preferred embodiment, the steroidal glycoside has the general structural formula (I) or a salt or ester thereof: 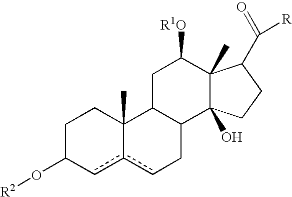
wherein
R=alkyl;
R1=H, alkyl, tiglyol, benzoyl or any other organic ester group;
R2 =H or one or more 6-deoxy carbohydrates, or one or more 2,6-dideoxy carbohydrates, or glucose molecules, or combinations thereof; and wherein the broken lines indicate the optional presence of a further bond between carbon atoms C4 and C5 or between carbon atoms C5 and C6.

According to another equally preferred embodiment, the plant extract is an extract from plants of the group comprising the genus Trichocaulon and the genus Hoodia, said extract having appetite suppressant activity. More preferably, said plant extract is selected from the group consisting of appetite suppressant Trichocaulon piliferum extracts, appetite suppressant Trichocaulon officinale extracts, appetite suppressant Hoodia currorii extracts, appetite suppressant Hoodia gordonii extracts, appetite suppressant Hoodia lugardii extracts and mixtures thereof.

The second aspect of the present the invention is the edible aqueous dispersion comprising one or more steroidal glycosides, preferably obtained from plants of the Asclepiadaceae family, and an emulsifier having a HLB (hydrophilic lipophilic balance) value between 7 and 30, wherein the ratio (w/w) of the steroidal glycosides to the emulsifier in said edible dispersion is less than 10:1. Preferably, said steroidal glycosides are dispersed to a mean particle size (D3,2) of less than 15 micrometer.

The edible aqueous dispersion can be used in food products, and this constitutes a third aspect of the present invention. Examples of such food products are beverages, snacks, bars, spreads, dressings, soups, etc., or meal replacement products, which can be used in the management of body weight or in the dietary control of obesity.

All amounts, parts, ratios and percentages used herein are by weight, unless otherwise specified.

While the above summarizes the present invention, it will become apparent to those skilled in the art that modifications, variations and alterations may be made without deviating from the scope and spirit of the present invention as described and claimed herein. The invention will now be further illustrated in the following, non-limiting examples.

### EXAMPLE 1

Hoodia Gordonii extract water dispersion process.

450 ml demineralised water was heated to 95° C. in a 500 ml beaker. The water was agitated using a Silverson mixer set at 5,500 rpm. Then, 1 g sodium stearoyl lactylate was added and the mixture was agitated until the stearoyl lactylate was completely dissolved. The shaft speed of the Silverson was then increased to 6,200 rpm and 5 g Hoodia gordonil extract (obtained according to the process of WO-A-98/46243) containing about 31% (w/w) steroidal glycosides was added and mixed until it was fully dispersed. The dispersion was then cooled below 40° C. by putting the beaker in an ice bath under while continuing mixing at 5,000 rpm. Demineralised water was added to a total volume to 500 ml.

### EXAMPLE 2

Particle sizes of aqueous dispersions of Hoodia gordonii extracts.

Similar Hoodia gordonii extract dispersions as in Example 1 were made by substituting for 1 g sodium stearoyl lactylate for 1 g Tween 60, 1 g Bolec MT or 1 g Sucrose ester L1695. Particle sizes were determined by means of laser diffraction using a Helium-Neon Laser Optical System. The results are shown below.

| | Emulsifier | Hoodia gordonii | Measured Mean |
|---|---|---|---|
| | content | content | Particle size |
| Emulsifier | (% w/w) | (% w/w) | (D3.2 in μm) |
| Sodium stearoyl | 0.2 | 1.0 | 0.7 |
| lactylate | | | |
| Bolec MT | 0.2 | 1.0 | 4.2 |
| Vitamin E TPGS | 0.2 | 1.0 | 7.4 |
| Sucrose ester L1695 | 0.2 | 1.0 | 6.2 |

The Hoodia gordonii extracts used in this example contained 31% (w/w) steroidal glycosides.

### EXAMPLE 3

Typical food composition comprising an aqueous dispersion of Hoodia gordonii.

The following edible composition in the form of a drink was prepared:

| Ingredient | Content (% w/w) |
|---|---|
| Sodium stearoyl lactylate | 0.20 |
| Hoodia gordonii extract | 0.20 |
| Canola oil | 5.00 |
| Skim milk powder | 6.75 |
| Sucrose | 5.00 |
| HM-Pectin | 0.45 |
| Acesulfame-K | 0.05 |
| Aspartame | 0.05 |
| Citric acid, 50% solution | 1.25 |
| Water, demineralised | 81.05 |

A 4,000 ml beaker was filled with 3,200 ml of the aqueous Hoodia gordonii dispersion containing 8 g Sodium stearoyl lactylate and 8 g Hoodia gordonii extract. The dispersion was reheated to 75° C. under moderate stirring. The vegetable oil was gently added and mixture was stirred for 2-3 minutes. Then, the skim milk powder, the pectin and the sugars/sweeteners as a powder blend were added and mixed for 5-8 minutes until all lumps had disappeared. During mixing the citric acid solution was slowly added to set the pH of the drink to 4.0. Finally, demineralised water was added to set the total amount to 4,000

### EXAMPLE 4

Drinks were prepared as described in example 3 with 1.0% (w/w) Hoodia gordonii extract and 0.2% (w/w) SSL. The drink was pasteurised at 78° C. and 150 bar for 40 s and transferred to bottles using hot fill at 75° C. The bottles were heat-sealed and stored at 2-8° C. After 98 days, the solution was still stable with no phase separations and a mean particle size (D3.2) of 5.0 μm.

### EXAMPLE 5

Formulations for 0%, 6& and 24% fat water continuous spreads with 0.5% Hoodia gordonii extract

| Ingredients | 0% | 6% | 24% |
|---|---|---|---|
| 0.25% (w/w) SSL/1.0% (w/w) Hoodia | 200 | 200 | 200 |
| gordonii extract dispersion | | | |
| Sun flour oil | 0 | 24 | 96 |
| Water | 177.95 | 155.15 | 84.45 |
| Gelatin (beef) | 5 | 4.5 | 4 |
| Starch, Remyline AP | 5 | 4.5 | 4 |
| Lactose | 4 | 3.8 | 3.5 |
| Lactic Acid | 0.3 | 0.3 | 0.3 |
| K Sorbate | 0.05 | 0.05 | 0.05 |
| Salt | 6.5 | 6.5 | 6.5 |
| Buttermilk Powder | 1 | 1 | 1 |
| Beta Carotene CWS 1% | 0.15 | 0.15 | 0.15 |
| Flavor and Vitamins | 0.05 | 0.05 | 0.05 |
| Total | 400 | 400 | 400 |

Preparation: the SSL and Hoodia gordonii extract were dispersed in water as described in Example 1 and heated to 90° C. Sun flour oil, gelatin, starch and water were added at 90° C. and mixed well using a turbomixer. The remaining ingredients were added under agitation and the mixture was cooled down to 20-25° C., poured in cups and refrigerated.

### EXAMPLE 6

Formulation for a creamy Italian dressing with 0.4% (w/w) Hoodia gordonii extract.

| Ingredients | Content (% w/w) |
|---|---|
| Soybean Oil | 44.5 |
| Water | 2.5 |
| Sugar | 4.5 |
| Vinegar 120 Grain | 3.0 |
| HVF Algin | 0.1 |
| Buttermilk Powder | 1.0 |
| Lactic Acid | 0.3 |
| Lemon Juice Cone | 0.4 |
| Salt | 2.5 |
| Minced Onion | 0.2 |
| Minced Garlic | 0.5 |
| Xanthan Gum | 0.1 |
| Red Bell Peppers | 0.2 |
| MSG | 0.1 |
| Spices | 0.1 |
| 0.25% (w/w) SSL/1.0% (w/w) Hoodia | 40.0 |
| gordonii extract dispersion | |
| Total | 100.0 |

Preparation: Combine ingredients under agitation and process through colloid mill.

It will be apparent that for commercialization the previously mentioned process steps may be up-scaled to the appropriate process and equipment sizes, types and standards practised in the particular or relevant food industry.

It should be understood that the specific forms of the invention herein illustrated and described are intended to be representative only and that certain changes may be made therein without departing from the clear teachings of the disclosure.

What is claimed is:

1. Process for preparing an edible aqueous dispersion of one or more steroidal glycosides, comprising the steps of:
(a) preparing a composition comprising at least about 1% by weight steroidal glycosides,
(b) dissolving an emulsifier having a HLB (hydrophilic lipophilic balance) value between about 7 and about 30 in a solvent, and
(c) thoroughly homogenising the composition comprising the steroidal glycosides for about 1 to 10 minutes in the solution of the solvent and the emulsifier at a temperature of about 20 to about 200° C. to obtain a dispersion, whereby said steroidal glycosides are dispersed to a mean particle size (D3,2) of less than about 15 micrometer, wherein the ratio (w/w) of the steroidal glycosides to the emulsifier in said edible dispersion is less than about 10:1

2. Process according to claim 1 further comprising cooling the solution after step (c) to a temperature below about 40° C.

3. Process according to claim 1, wherein the solvent is selected from the group consisting of water, aqueous sugar solution, protein solutions, and food-grade oils.

4. Process according to claim 2 wherein the food-grade oils are triglycerides.

5. Process according to claim 1, wherein the emulsifier is selected from the group consisting of stearoyl lactylates, polysorbates, lecithin, sucrose esters and mixtures thereof.

6. Process according to claim 1, wherein the steroidal glycoside is obtained from plants of the Asclepiadaceae family.

7. Process according to claim 6, wherein the steroidal glycoside is obtained from plants of the Hoodia genus.

8. Process according to claim 7, wherein the plant is selected from Hoodia gordonii, Hoodia curroril subsp. currorril and Hoodia currorii subsp. Tugardii.

9. Process according to claim 8, wherein the plant is Hoodia gordonii.

10. Process according to claim 8, comprising one or more steroidal glycosides having the formula: 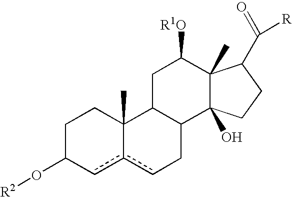
wherein R=alkyl;
R1=H, alkyl, tiglyol, benzoyl or any other organic ester group;
R2 =H or one or more 6-deoxy carbohydrates, or one or more 2,6-dideoxy carbohydrates, or glucose molecules, or combinations thereof; and wherein the broken lines indicate the optional presence of a further bond between carbon atoms C4 and C5 or between carbon atoms C5 and C6, or mixtures thereof.

11. Process according to claim 1, wherein the composition in step (a) is a plant extract.

12. Process according to claim 11, wherein the extract comprises at least about 10% of steroidal glycosides.

13. Process according to claim 1, wherein the steroidal glycoside has the formula:

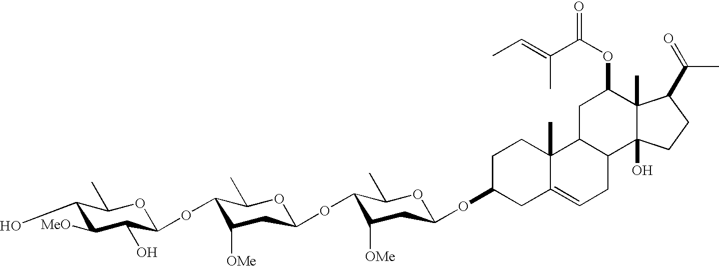

14. An edible aqueous dispersion comprising one or more steroidal glycosides and an emulsifier having a hydrophilic lipophilic balance value between about 7 and about 30, wherein the ratio (w/w) of the steroidal glycosides to the emulsifier in said edible dispersion is less than about 10:1.

15. Edible dispersion according to claim 14 wherein said steroidal glycosides are dispersed to a mean particle size (D3,2) of less than about 15 micrometer.

16. Food product comprising the dispersion according to claim 14.

17. Food product according to claim 16, in the form of a beverage, snack, bar, spread, dressing, soup or meal replacement product.

18. Process for preparing a food product comprising incorporating into the food product the edible dispersion according to claim 14.

19. A method of managing body weight or of dietary control of obesity, the method comprising ingesting the food product according to claim 15.
